(19) United States
(12) Patent Application Publication
(10) Pub. No.: US 2007/0225667 A1
(43) Pub. Date: Sep. 27, 2007

(54) Urine collection receptacle

(76) Inventor: Edgar A. Otto, Boca Raton, FL (US)

Correspondence Address: CHRISTOPHER & WEISBERG, P.A., 200 EAST LAS OLAS BOULEVARD, SUITE 2040, FORT LAUDERDALE, FL, 33301, US

(21) Appl. No.: 11/725,791

(22) Filed: Mar. 20, 2007

Related U.S. Application Data
(60) Provisional application No. 60/785,191, filed on Mar. 22, 2006.
(60) Provisional application No. 60/864,954, filed on Nov. 8, 2006.

(51) Int. Cl. A61M 1/00

(52) U.S. Cl. 604/317

(57) ABSTRACT

The present invention includes a self-contained, rechargeable battery powered, compact urine collection system adapted to receive and reliably store urine from a user. The urine collection system may be configured differently for male and female users or may include a collection receptacle adapted to accommodate both male and female users. The collection system may further provide a collection reservoir expandable in a single direction upon filling.

### CROSS-REFERENCE TO RELATED APPLICATION

This application is related to and claims priority to U.S. Provisional Patent Application Ser. No. 60/785,191, filed Mar. 22, 2006, entitled APPARATUS FOR URINE COLLECTION, and U.S. Provisional Patent Application Ser. No. 60/864,954, filed Nov. 8, 2006, entitled METHODS AND APPARATUS FOR A URINE COLLECTION BAG, both of which are incorporated herein by reference in their entirety.

### STATEMENT REGARDING FEDERALLY SPONSORED RESEARCH OR DEVELOPMENT

n/a

### FIELD OF THE INVENTION

The present invention is generally directed to urine collection devices, and more particularly, to compact, portable urine collection methods and systems for discretely collecting and storing a high volume of urine from a user.

### BACKGROUND OF THE INVENTION

Urinary incontinence (UI) is defined as unintentional loss of urine that is sufficient enough in frequency and amount to cause physical and/or emotional distress in the person experiencing it. This condition is very prevalent. As of 2006, over 15 million people in the United States alone are incontinent and the number is growing with the aging of the “baby boomer” generation. One in every four women between the ages of 30 and 59 has experienced an episode of UI. Fifty percent or more of the elderly persons living at home or in long-term care facilities are incontinent. Thus the problem is significant, as are the related costs. In the United States alone, over $28 billion is spent per year on incontinence-related care via community-based programs, at home, and in long-term care facilities. More than $1.1 billion is spent every year on disposable products for adults.

UI is a voiding dysfunction that, as mentioned above, affects over 15 million people in America alone, with the highest prevalence in the elderly in both community and institutional settings. The high prevalence of UI and its significant adverse physical, psychological, and financial effects clearly justify more aggressive efforts to identify, evaluate, and treat UI in all settings. Growing evidence indicates that appropriate management can reduce the morbidity and cost of UI, particularly in institutionalized populations.

Although the prevalence of UI increases with age, UI is not considered a normal part of the aging process. Reported prevalence rates of UI vary considerably, depending on the population studied, the definition of UI, and how the information is obtained. Among the population between 15 and 64 years of age, the prevalence of UI in men ranges from 1.5 to 5 percent and in women from 10 to 30 percent. Women are affected by the disorder more frequently than are men; one in 10 women under age 65 suffer from urinary incontinence. Older Americans, too, are more prone to the condition with twenty percent of Americans over age 65 being incontinent. For non-institutionalized persons older than 60 years of age, the prevalence of UI ranges from 15 to 35 percent, with women having twice the prevalence of men. Between 25 and 30 percent of those identified as incontinent have frequent incontinence episodes, usually daily or weekly. Survey data from caregivers of the elderly show that approximately 53 percent of the homebound elderly are incontinent. A random sampling of hospitalized elderly patients identified 11 percent as having persistent UI at admission and 23 percent at discharge.

UI is generally recognized as one of the major causes of institutionalization of the elderly. Among the more than 1.5 million nursing facility residents, the prevalence of UI is 50 percent or greater, with the majority of nursing home residents having frequent UI. The annual incidence of UI in nursing home residents who are admitted continent was recently reported to be 27 percent and is higher in males; it is strongly associated with dementia, and the inability to walk and transfer independently.

There are five generally recognized categories of urinary incontinence: overflow, stress, urge, functional, and reflex. In some cases, an individual may develop short-term or acute incontinence. Acute incontinence may occur as a symptom or by-product of illness, as a side effect of medication, or as a result of dietary intake. The condition is typically easily resolved once the cause is determined and addressed.

Overflow incontinence is caused by bladder dysfunction. Individuals with this type of incontinence have an obstruction to the bladder or urethra, or a bladder that does not contract properly. As a result, their bladders do not empty completely, and they have problems with frequent urine leakage. Stress incontinence occurs when an individual involuntarily loses urine after pressure is placed on the abdomen (i.e., during exercise, sexual activity, sneezing, coughing, laughing, or hugging). Urge incontinence occurs when an individual feels a sudden need to urinate, and cannot control the urge to do so. As a consequence, urine is involuntarily lost before the individual can get to the toilet. Individuals who have control over their own urination and have a fully functioning urinary tract, but cannot make it to the bathroom in time due to a physical or cognitive disability, are functionally incontinent. These individuals may suffer from arthritis, Parkinson's disease, multiple sclerosis, or Alzheimer's disease. Individuals with reflex incontinence lose control of their bladder without warning. They typically suffer from neurological impairment. Other factors can cause incontinence such as decreased mobility, cognitive impairment or medications.

UI imposes a significant psychosocial impact on individuals, their families, and caregivers. UI results in a loss of self-esteem and a decrease in ability to maintain an independent lifestyle. Dependence on caregivers for activities of daily life increases as incontinence worsens. Consequently, excursions outside the home, social interaction with friends and family, and sexual activity may be restricted or avoided entirely.

In addition to UI, according to the U.S. National Spinal Cord Injury Database, the number of people in the United States who are alive and have SCI has been estimated to be up to 288,000 persons. It is estimated that the annual incidence of SCI patients who survive their accident is approximately 40 cases per million in the U.S. or approximately 11,000 new cases each year. After a spinal injury, the individual may not have as much control over the “urge” to urinate when their bladder is full, or have a severely limited amount of time before urination begins. They also may not have voluntary control of their bladder and sphincter muscles thereby requiring more frequent and urgent urinations throughout the day. Individuals with SCI face a high risk for urinary tract infection (UTI). In fact, complications due to UTI are the number one medical concern and more likely to affect SCI individuals' overall health and increase health care costs. Nearly all patients with spinal injuries have little sensation and may have little to no warning of the need to urinate. If no convenient means of voiding is available, these patients risk incontinence and unintentionally leak urine. If not addressed quickly this residual moisture may create sores, swelling, inflammation, and possible infection requiring additional care.

Removal of urine from incapacitated humans has been undertaken using a variety of devices and methods with limited success. For instance, diapers have been used to capture urine from patients. While diapers may collect most of the urine produced by a patient, diapers hold moisture against the skin, which can cause rashes on the patient. In addition, diapers must be changed frequently to function adequately and avoid leakage. The idea of wearing a diaper, having to remain in a soiled diaper, and the difficulties associated with changing and disposing of soiled diapers involve significant psychological, emotional, and logistical drawbacks for users of diapers. For at least these reasons, diapers are not a desirable choice for collecting urine from a patient.

Another device commonly used to collect urine from incapacitated patients who are unable to use conventional toilets is a bedpan. Bedpans have been used successfully but produce undesirable odors and are, at times, unsightly. In addition, bedpans often require the assistance of a nurse to use. While a nurse is respectful of a patient's privacy, nurses often make patients uncomfortable. Use of bedpans is also limited to use with beds that are adapted for their use. Thus, bedpans are not portable and are not capable of being used in a variety of locations.

Catheters and urine condoms are also commonly used to remove urine from men. Catheters are typically composed of small diameter tubing that is placed inside the urethra of a patient. Urine condoms are worn on the exterior of the penis. While catheters and urine condoms may be efficient at removing urine from men, these devices frequently cause infection and maybe painful or uncomfortable to insert or wear.

A class of urine collection devices in addition to those discussed above include urine suction devices. Urine suction devices transport urine from a patient to a tank or reservoir using pumps. For instance, U.S. Pat. No. 6,311,339 issued to Kraus, which is incorporated herein by reference, is directed to such a suction collector that receives urine in a well accessed by a flexible urine collection conduit. The suction collector includes a vacuum for transporting urine from the well to a separate tank via the conduit. The suction collector of Kraus is activated once a urine collection receptacle is sealed against a patient's skin surface and the pump is actuated.

While such suction collectors overcome some of the disadvantages of the diaper and the bedpan, such suction collectors include a number of drawbacks. For example, the Kraus device which is not portable, requires a vacuum coupling with the skin of the user that must be adjusted, a medical attendant to operate the device, a large separate tank for separating urine from air, and a separate AC powered vacuum source. In addition to these requirements, such prior art devices generally lack safety features that would avoid injury to the users. For example, there is nothing to shut down the Kraus suction collector in the case of too much vacuum pressure or a filled tank. Further, prior art suction collectors such as the Kraus device are not suitable for use in environments other than a hospital. The pumps are loud and the system must remain upright and generally stationary to prevent urine spills and damage to the pump.

In view of the above limitations, it would be desirable to provide an apparatus for removing urine that provides (i) diminished risk of skin irritation, rashes and sores because moisture content near the user is reduced; (ii) reduction in the high incidence of infection and resulting costs of treatment because the device is external and self-applied; (iii) sufficient volume storage for a day's worth of urine before discarding (as opposed to other alternative that require immediate disposal); (iv) reduction in the instances of “slip and fall” accidents, as the device will eliminate the necessity of the users to transfer locations or rush to the bathroom; and (v) ease of use and maintenance compared to other prior art devices.

### SUMMARY OF THE INVENTION

The present invention may include a self-contained, rechargeable battery powered, compact urine collection system adapted to receive and reliably store urine from a user. The urine collection system may be configured differently for male and female users or may include a collection receptacle adapted to accommodate both male and female users. The collection receptacle may include a channel adapted to receive and direct a stream of urine and prevent or minimize splash back. The collection receptacle may be further shaped and sized to prevent or minimize splash back and to both prevent overflow and spillage. The urine collection receptacle may be coupled to a reservoir using a conduit. A check valve may be positioned along the conduit to prevent urine from flowing back into the collection receptacle and/or out of the reservoir. The reservoir may be compact, portable, and releasably coupled to the conduit. The reservoir may include an air pressure relief valve and a drainage outlet for emptying the reservoir into a conventional toilet or other appropriate repository.

The collection receptacle, the conduit, and/or the reservoir may be disposable and be made commercially available as a separate kit for use with the urine collection system of the present invention. In some embodiments, existing commercially available urinary drainage and/or medical fluid reservoirs may be used as a reservoir. The urine collection system may also include a pump coupled to the conduit for pumping urine from the urine collection receptacle to the reservoir. A pump may be used that is capable of pumping urine without contacting the urine. For example, in some embodiments, the pump may be a peristaltic pump. In some embodiments, the pump may be adapted to avoid creating an air entrained liquid, e.g., foam. In some embodiments, the pump may be adapted to operate silently or nearly silently relative to ambient noise levels. In addition, the conduit may include a valve (e.g., a check valve) and/or a muffler chamber that is adapted to function as a muffler to further reduce noise generated from pumping urine and/or air. The conduit itself may be constructed from a material (e.g., rubber hose) adapted to absorb vibrations that may be generated from pumping urine and/or air. Further, in some embodiments, the conduit may be adapted to advantageously collapse as a safety feature if more than a threshold amount of vacuum pressure is generated within the conduit.

The urine collection system of the present invention may include a discrete, compact, weather-proof enclosure adapted to contain and protect the entire system. For example, the enclosure may be adapted to resemble a black leather, laptop-sized brief case with either hard or soft sides. The enclosure may include a control and status panel that allows the user to operate the urine collection system and receive status information about, e.g., the fullness of the reservoir, remaining battery power, and/or the operation of the pump. The enclosure may be adapted to further suppress any noise that may be generated from pumping urine and/or air. The enclosure may include a convenient swing-out compartment for storing and accessing the collection receptacle and conduit which may be adapted to retractably coil from the compartment. The enclosure may include side and/or end doors or access panels for accessing the reservoir and/or drainage outlet.

In some embodiments, the enclosure may include one or more sensors adapted to activate the urine collection system when the collection receptacle is removed from the enclosure or the enclosure is opened to access the collection receptacle. The enclosure may further include one or more sensors adapted to de-activate the urine collection system when the collection receptacle is replaced, the swing-out compartment is closed, the reservoir has reached a threshold level of fullness, and/or if the reservoir access door is opened. In some embodiments, the enclosure may be adapted to be mounted on a powered chair, a mechanical wheelchair, an airplane/bus/train/automobile seat, and/or a scooter such that a mounted urine collection system does not increase the overall width of the chair, seat and/or scooter. In addition, the enclosure may be further adapted to be mounted either directly, or through the use of a frame, to a bed, gurney, seat, and/or intravenous fluids cart.

An advantage of the present invention is that the urine collection system is discrete, compact, self-contained, quite, portable, able to be operated in different orientations (e.g., inverted, on its side, etc.), and able to be operated while in motion (e.g., while being jarred, bumped, etc.). The present invention enables users to urinate in any location with comfort and without assistance. For example, the present invention allows a user to urinate into the collection receptacle, under a blanket, while seated on an airplane without nearby passengers being aware of what the user is doing. An advantage of embodiments of the present invention that use a peristaltic pump is that the urine collection system is a closed system wherein every portion of the urine collection device that contacts urine may be easily removed from the device and disposed. Thus, this system is easy to clean.

Another advantage of embodiments of the present invention is that sensors may be used to precisely monitor the reservoir to accurately determine the volume of urine contained in the reservoir for medical monitoring purposes and to inform the user. Another advantage of the present invention is that the urine collection system may include either a unisex urine collection receptacle, or interchangeable receptacles separately configured for males and/or females. Yet another advantage of the present invention is that the urine collection system prevents urine from flowing backwards through the conduit back into the urine collection receptacle after urine has entered the conduit. Another advantage of the present invention is that the system includes an automatic shutdown safety feature in the event the reservoir becomes full or becomes dislodged from the pump coupling. These and other features and advantages of the present invention will become apparent after review of the following drawings and detailed description of the disclosed embodiments.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a perspective view of an embodiment of a urine collection system in accordance with the present invention;

FIG. 2 is a side view of an embodiment of a urine collection system in accordance with the present invention;

FIG. 3 is an additional side view of an embodiment of a urine collection system in accordance with the present invention;

FIG. 4 is a front view of an embodiment of a urine collection system in accordance with the present invention;

FIG. 5 is a rear view of an embodiment of a urine collection system in accordance with the present invention;

FIG. 6 is a top view of an embodiment of a urine collection system in accordance with the present invention;

FIG. 7 is a bottom view of an embodiment of a urine collection system in accordance with the present invention;

FIG. 8 is an additional perspective view of an embodiment of a urine collection system in accordance with the present invention;

FIG. 9 is a perspective view of an embodiment of a urine collection system with a swing-out compartment opened in accordance with the present invention;

FIG. 10 is a perspective view of an embodiment of a urine collection system removed from and shown next to a portion of an enclosure in accordance with the present invention;

FIG. 11 is a close-up perspective view of an embodiment of a control/status panel of a urine collection system in accordance with the present invention;

FIG. 12 is a perspective view of an embodiment of a urine collection system without a portion of an enclosure in accordance with the present invention;

FIG. 13 is an exploded perspective view of an embodiment of a urine collection system in accordance with the present invention;

FIG. 14 is a perspective view of an embodiment of a pump suitable for use with a urine collection system in accordance with the present invention;

FIG. 15 is an exploded perspective view of an embodiment of a pump suitable for use with a urine collection system in accordance with the present invention;

FIGS. 16-22 are respective views of an embodiment of a collection receptacle assembly suitable for use with a urine collection system in accordance with the present invention.

FIG. 23 is a cross-sectional view of an embodiment of a collection receptacle assembly suitable for use with a urine collection system in accordance with the present invention;

FIG. 24 is another cross-sectional view of an embodiment of a collection receptacle assembly suitable for use with a urine collection system in accordance with the present invention;

FIG. 25 is a close-up cross-sectional perspective view of a portion of the collection receptacle assembly of FIG. 24;

FIG. 26 is a front view of an embodiment of a fluid collection reservoir in accordance with the present invention;

FIGS. 27 and 28 are side views of an embodiment of a fluid collection reservoir in accordance with the present invention;

FIG. 29 is a cross sectional view of an embodiment of a fluid collection reservoir in accordance with the present invention;

FIG. 30 is a perspective view of an embodiment of a urine collection system mounted on a powered wheelchair in accordance with the present invention;

FIG. 31 is a perspective view of an embodiment of a urine collection system supported by a frame mounted on a bed in accordance with the present invention; and

FIG. 32 is a schematic block diagram of an embodiment of a urine collection system control circuit in accordance with the present invention.

### DETAILED DESCRIPTION OF THE INVENTION

The present invention provides a urine collection system for collecting urine from users that is discrete, compact, self-contained, quite, portable, includes fail-safe safety measures, is adapted to be operated in different orientations (e.g., inverted, on its side, etc.), is adapted to be operated while in motion (e.g., while being jarred, bumped, etc.), and is adapted to be used without assistance. A urine collection system in accordance with the present invention may be used to collect urine from humans and animals alike. More specifically, the disclosed urine collection system may be used to collect urine from bedridden patients, wheelchair-bound users, users who may have limited mobility, users who have various forms of urinary incontinence, people unable to use conventional toilets, users who are traveling, and others. The present urine collection system may also be used to collect urine from patients in various positions, such as, but not limited to, a seated position, a standing position, a prostrate position, a reclined position, an upright position, and other positions.

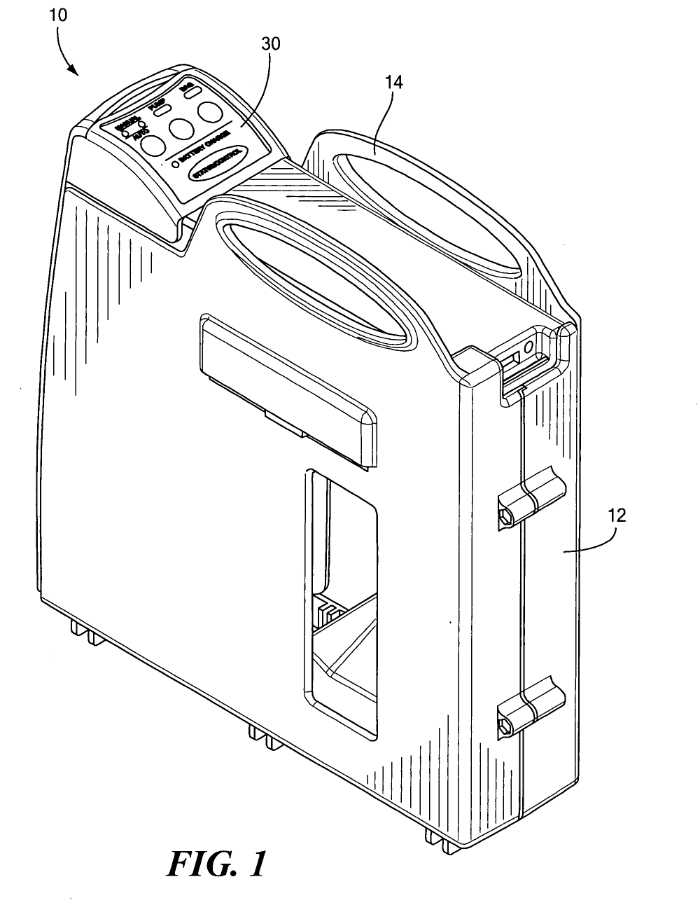

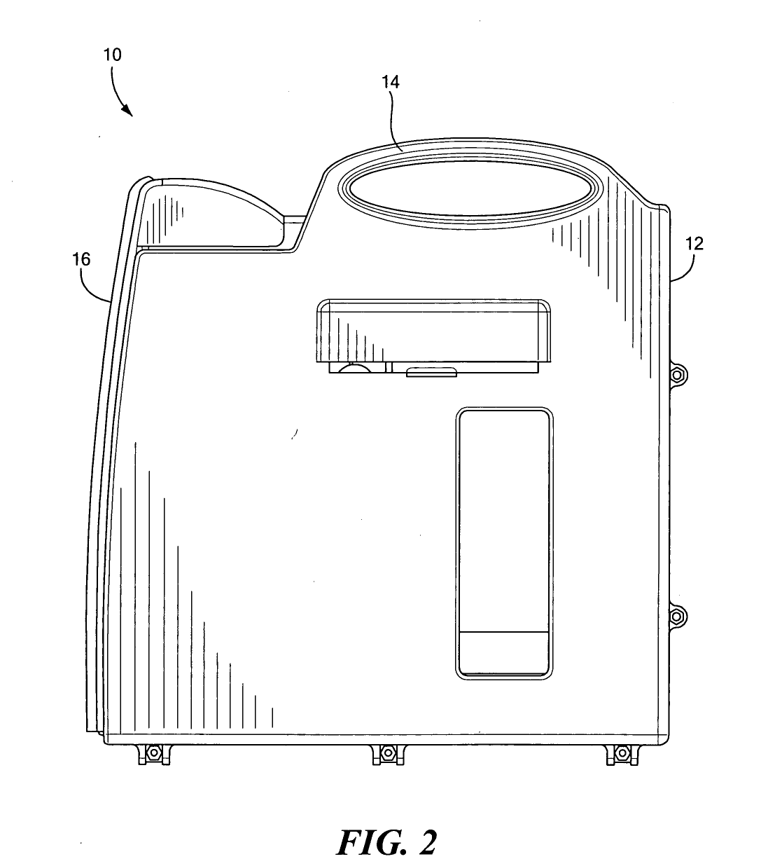

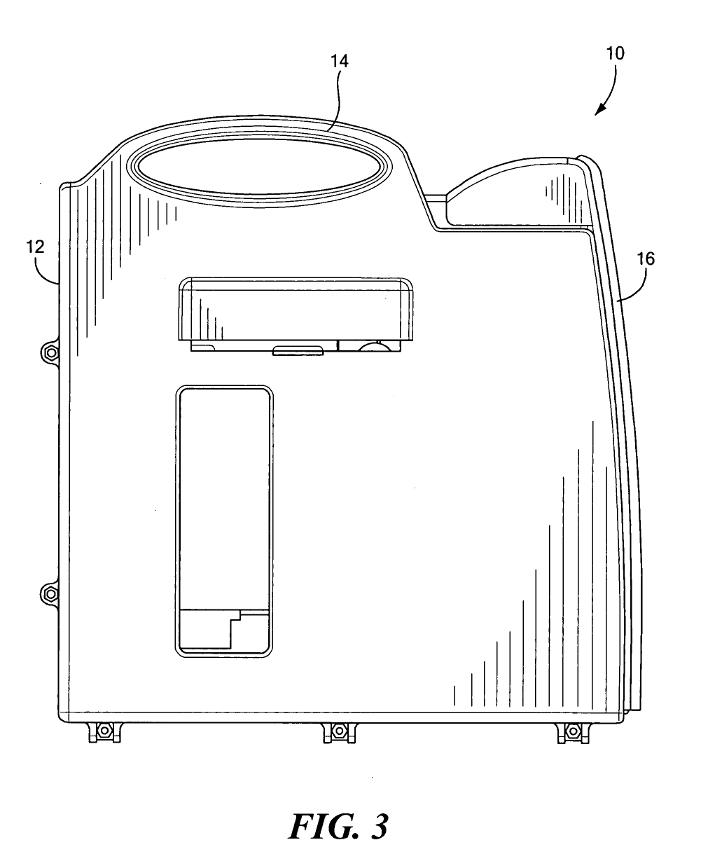

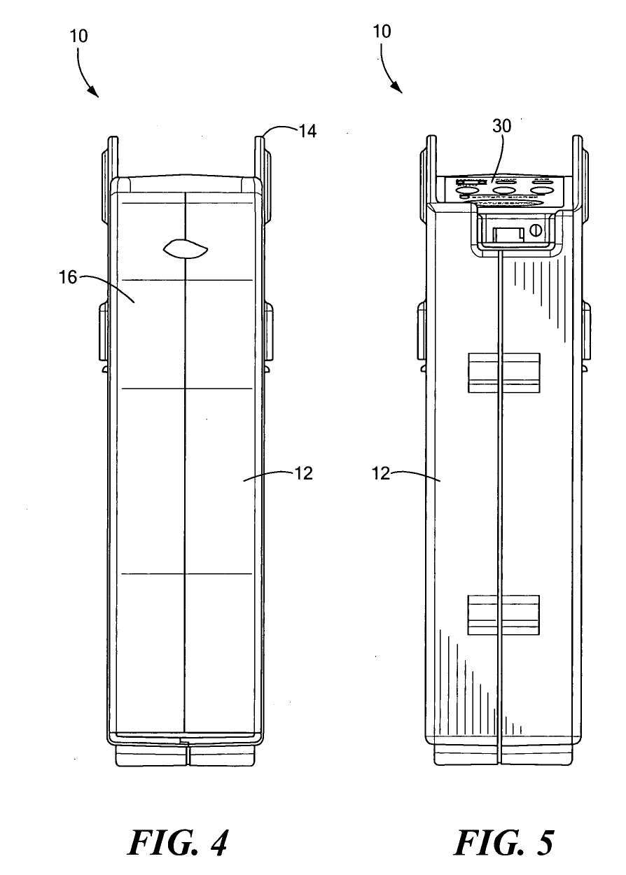

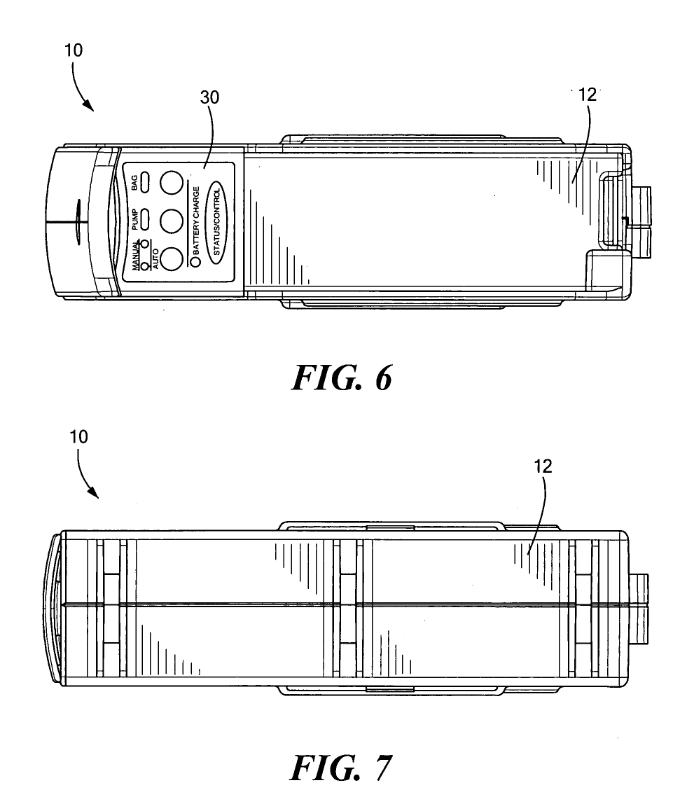

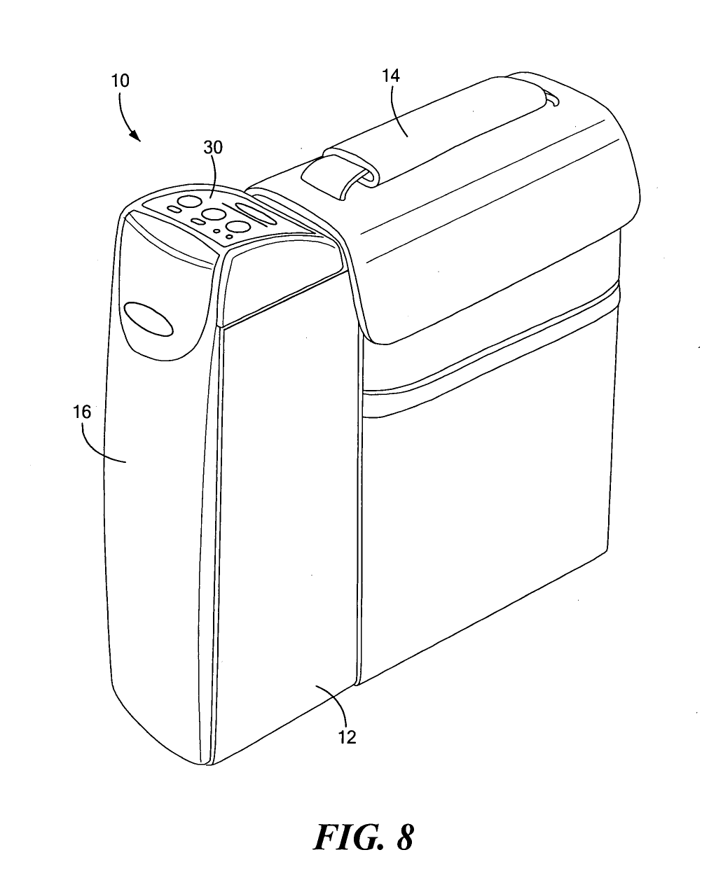

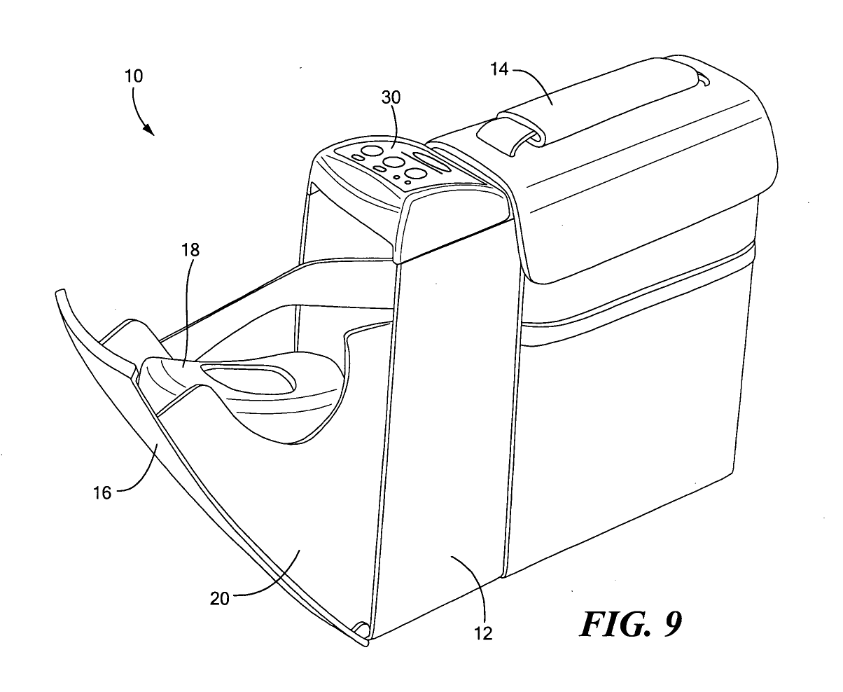

Turning now to the drawings, embodiments of the present invention are described in detail. Now referring to FIGS. 1-8, a urine collection system 10 is shown, where the collection system may include an enclosure 12 surrounding a rigid but light weight frame and defining an interior cavity therein. The enclosure 12 may include one or more top handles 14 and/or side pockets. The top of the enclosure 12 may further include material and/or a flap (e.g., including Velcro®) for suspending or hanging the urine collection system 10 from the arm of a powered scooter or a swing-arm mounted to a mechanical wheelchair, as will be described in more detail below. The enclosure 12 includes and/or provides a door 16 movably coupled to the enclosure 12, providing access to the interior cavity. The door 16 may provide a swing-out compartment that stores a urine collection receptacle or cup 18. The enclosure 12 also includes and/or provides removable and/or open-able side access panels or doors. The enclosure 12 may be manufactured from a wide variety of materials that are easy to clean and generally waterproof or water resistant (such as, for example, plastic, synthetic leather or rubber, vinyl, etc.) such that the urine collection system 10 is weather-proof. The materials for the component parts of the present invention may be chosen to minimize the overall weight of the system while still providing durability and reliability. For example, light weight, durable plastics may be used as much as possible and aluminum may be used where metal parts are desirable or appropriate. The overall appearance of the urine control system is that of a small, nondescript, slim profile, briefcase.

Turning to FIG. 9, a perspective view of an embodiment of a urine collection system 10 with the door 16 opened is depicted. One or more protruding sidewalls 20 may extend from the door 16 to provide a swing-out compartment able to removably receive the collection receptacle 18. The positioning of the swing-out compartment provides easy, ergonomic access to, and a view of, the collection receptacle 18 by a user seated adjacent the urine collection system 10. The swing-out compartment and/or the enclosure 12 may include one or more sensors for detecting that the swing-out compartment has been opened. In response to the sensors detecting that the swing-out compartment has been opened, the urine collection system 10 may automatically activate a vacuum pump to create vacuum pressure within or air flow into the urine collection receptacle 18. In some embodiments, the swing-out compartment may include a catch that may be released to open the compartment. A sensor may be used to detect that the catch has been released and the urine collection system 10 may automatically activate the vacuum pump based on the release of the catch. Alternatively, the presence of the collection receptacle 18 may be monitored by a sensor that sends a signal to a control circuit of the urine collection system 10 to trigger activation of the vacuum pump based on removal of the collection receptacle 18 from the swing-out compartment. In some embodiments, the removable and/or open-able side access panels or doors may also include sensors to detect that the access panels have been opened. The urine collection system 10 may be adapted to shut-down or de-activate the pump in response to receiving a signal from such a sensor indicating that an access panel has been opened. Any number of different sensors may be used in the various embodiments described above. For example, optical sensors, pressure sensors, contact switches, inductive sensors, capacitive sensors, reflective sensors, etc. may be used. In some embodiments, sensors may be used to detect that a reservoir coupled to the collection receptacle 18 is nearly full or has become disconnected from the pump.

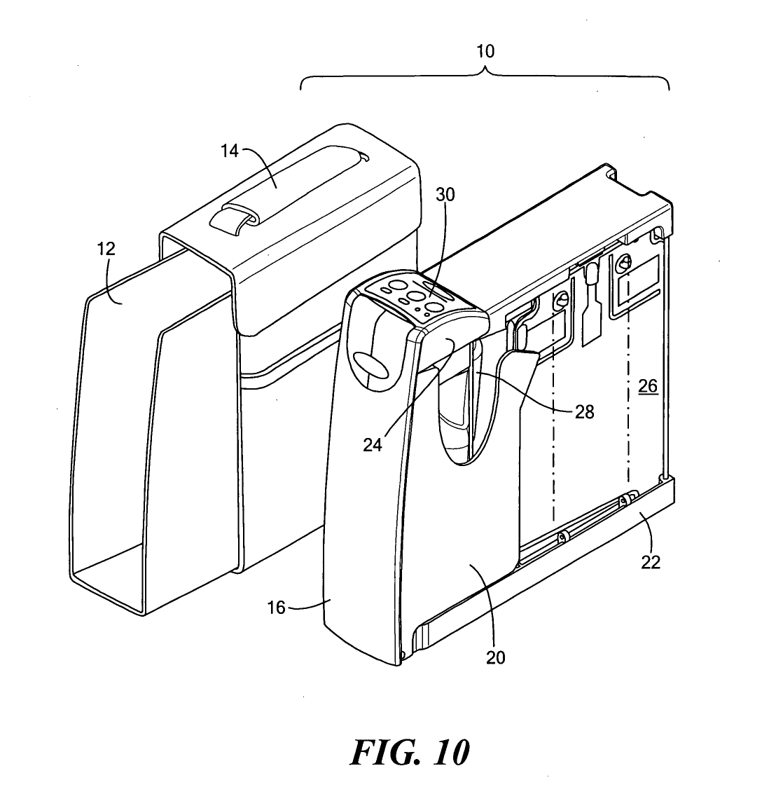

Turning to FIG. 10, the urine collection system 10 is depicted removed from, and beside, the enclosure 12. A rigid frame 22 is visible comprising a lower base plate, an upper top plate, and wire rails connecting the base plate and top plate. Mounted to the underside of the top plate, an inner enclosure 24 that houses the control circuitry and the pump is visible. Mounted to the inner enclosure 12, a reservoir 26 hangs down toward the base plate. The reservoir 26 depicted is empty and thus, is flat. However, as the reservoir 26 is filled with urine, the reservoir 26 is adapted to expand to fill the empty space between the base and top plates.

In some embodiments, the reservoir 26 includes a drainage tube to facilitate emptying the urine from the reservoir 26. In some embodiments, the pump may be operated in a drainage mode to help push or flush the urine out of the reservoir 26. The reservoir 26 may include a vent to allow trapped air to escape. In some embodiments the reservoir 26 may include paneled sides and or pleats to allow for expansion and storage of more urine. In some embodiments, the reservoir 26 may be adapted to hold one or more days worth of urine (e.g., approximately 1 to 2 liters). In some embodiments the system may include an auxiliary reservoir that may be used if the primary reservoir 26 becomes full. For example, the control circuit may cause the pump to automatically switch over to using the auxiliary reservoir 26 if the control circuit receives a signal from a sensor monitoring the primary reservoir 26 indicating that the primary reservoir 26 is full.

Referring again to FIG. 10, a portion of clear conduit tubing 28 that connects the collection receptacle 18 to the pump is visible protruding out of the inner enclosure 24. The conduit tubing 28 may be a pre-coiled (or a straight) length of about 0.25″ (e.g., approximately 0.125″ to approximately 0.5″) inner diameter tubing manufactured from a durable, clear material. In some embodiments, approximately 25″ to approximately 32″ of tubing may be used. At the end of the conduit, just before entering the pump, a soft, flexible material (e.g., rubber) may be inserted in-line with the conduit that is adapted to absorb percussive forces generated by the pump and the fluid flow. This percussion relief tube disposed inline between the conduit and the pump at the pump helps to significantly reduce the operating noise level of the system.

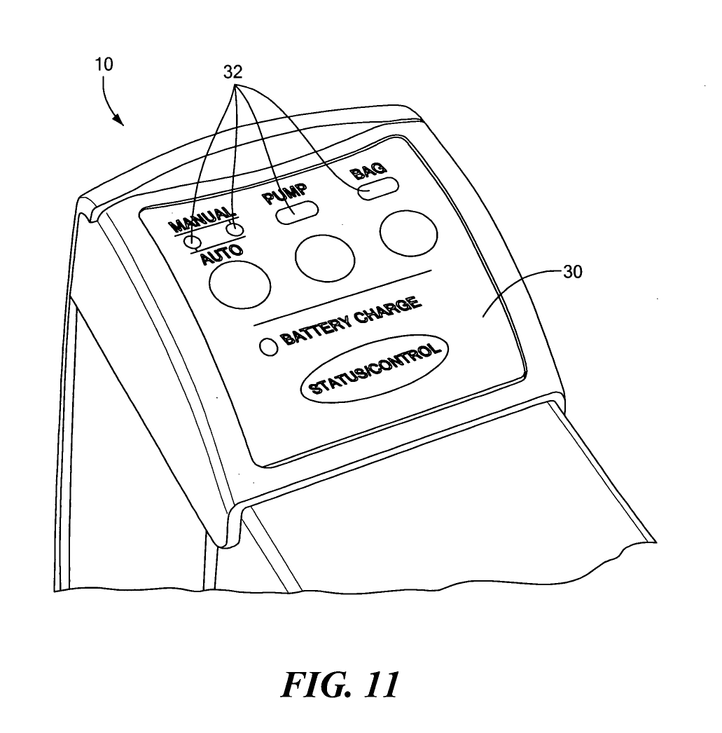

In addition, the enclosure 12 includes and/or provides an opening or surface for a status/control panel 30 on the top surface of the urine collection system 10. FIG. 11 depicts a close-up perspective view of an example embodiment of a status/control panel 30 that provides easy, ergonomic access to, and a view of, the panel by a user seated adjacent the urine collection system 10. The status/control panel may include a plurality of status indicators 32 that provide a visual, audio, or tactile indication of a condition of one or more components of the collection system. In particular, the status indicators 32 may convey information related to the pump (such as ON or OFF), a level of fluid within the reservoir 26, etc. The status indicators 32 may be coupled to or otherwise in communication with the one or more sensors described herein. In a particular embodiment, the status indicators 32 may include a number of lights that indicate a condition of the urine collection system 10. For example, when lit, the light labeled “AUTO” indicates that the urine collection system 10 is in an automated operating mode wherein the pump will turn on when, for example, the collection receptacle 18 is removed from the swing-out compartment and wherein the pump will turn off when, for example, the collection receptacle 18 is returned to the swing-out compartment. When lit, the light labeled “MANUAL” indicates that the urine collection system 10 is in a manual operating mode wherein the pump will only turn on if a power button is pressed and wherein the pump will turn off if the power button is pressed again. In normal operation, only one of the AUTO and MANUAL lights may be lit at a time. When lit, the light labeled “PUMP” indicates that the pump is activated. When not lit, the PUMP light indicates that the pump is not active. Alternatively and/or in addition, a blinking or flashing PUMP light may indicate that a maximum or threshold run-time of the pump has been exceeded, and as a result, the pump has been turned off automatically. When lit, the light labeled “RESERVOIR” indicates that the reservoir 26 has reached a predefined threshold level of fullness and should be drained, for example, before the system is used again or after the next use. When not lit, the RESERVOIR light indicates that the reservoir 26 contains less than the threshold level of urine. Alternatively and/or in addition, the RESERVOIR light may blink to indicate that the reservoir has reached a predetermined threshold and should be emptied, and further, the RESERVOIR light may be lit continuously to indicate the reservoir has reached its maximum capacity and as a result, the pump has been disabled. In some embodiments, multi-color LEDs may be used, for example, to indicate additional information. For example, the RESERVOIR light may be a tri-color light with four possible states that each indicate different levels of reservoir 26 fullness: off may indicate that a reservoir 26 is empty or nearly empty; green may indicate that the reservoir 26 is less than 75% full; yellow may indicate that the reservoir 26 is more than 75% full; and red may indicate that the reservoir 26 is completely full (or not present or the drain is open) and operation of the system has been suspended until the reservoir 26 is drained, replaced, or closed. Likewise, the light labeled “BATTERY” may indicate the level of energy remaining in the battery and the light labeled “CHARGE” may indicate that the battery is being recharged. The three example buttons depicted may be used for such things as switching between automatic and manual operating modes, to control the pump in the manual operating mode, and to silence audible status signals such as a beeping tone alarm indicating the reservoir 26 is full.

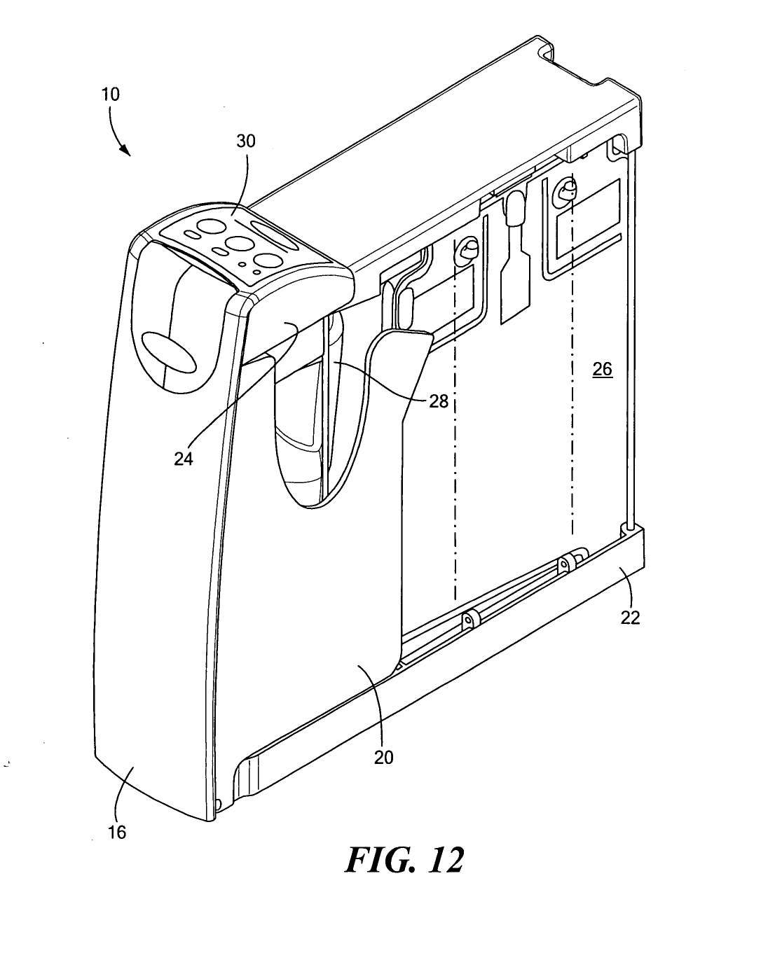

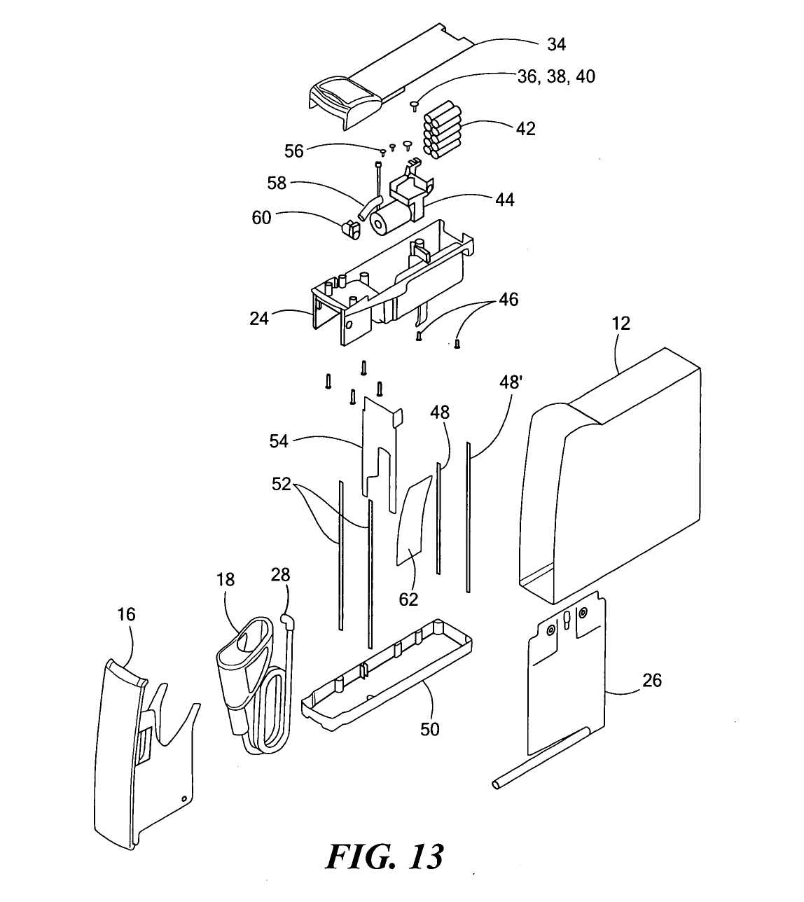

Turning to FIGS. 12 and 13, a line drawing perspective view and an exploded view of an example embodiment of a urine collection system 10 are depicted, respectively. The urine collection system 10 includes a control assembly 34, pump grommets 36, washers 38, fasteners 40, rechargeable batteries 42, pump assembly 44, fasteners 46, rails 48, 48′, enclosure 12, reservoir 26, base plate/drip tray 50, door/swing-out compartment 16, collection receptacle 18, conduit tubing 28, rails 52, conduit tubing barrier 54, inner enclosure 24, fastener 56, pump percussion relief tube 58, reservoir inlet connector 60, and sensor flap 62. These components are assembled as depicted in FIGS. 12 and 13. Sensor flap 62 may be adapted to rest against the reservoir 26 and as the reservoir 26 fills, the orientation or angle of flap 62 changes. A sensor (not shown) may be disposed so as to detect the orientation of the flap 62 and send signals to the control circuit indicating the fullness of the reservoir 26. In alternative embodiments, other methods of sensing the fullness of the reservoir 26 may be used. In particular, embodiments that are adapted to function independently of the orientation of the system (e.g., do not rely on gravity), may use sensors adapted to indicate the fullness of the reservoir 26 in any orientation.

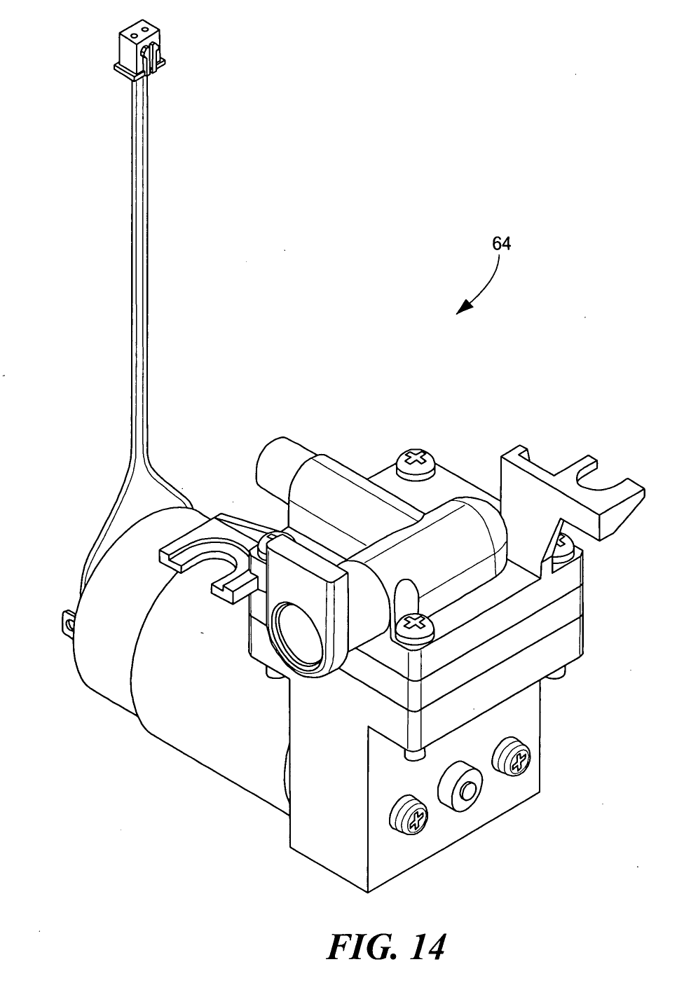

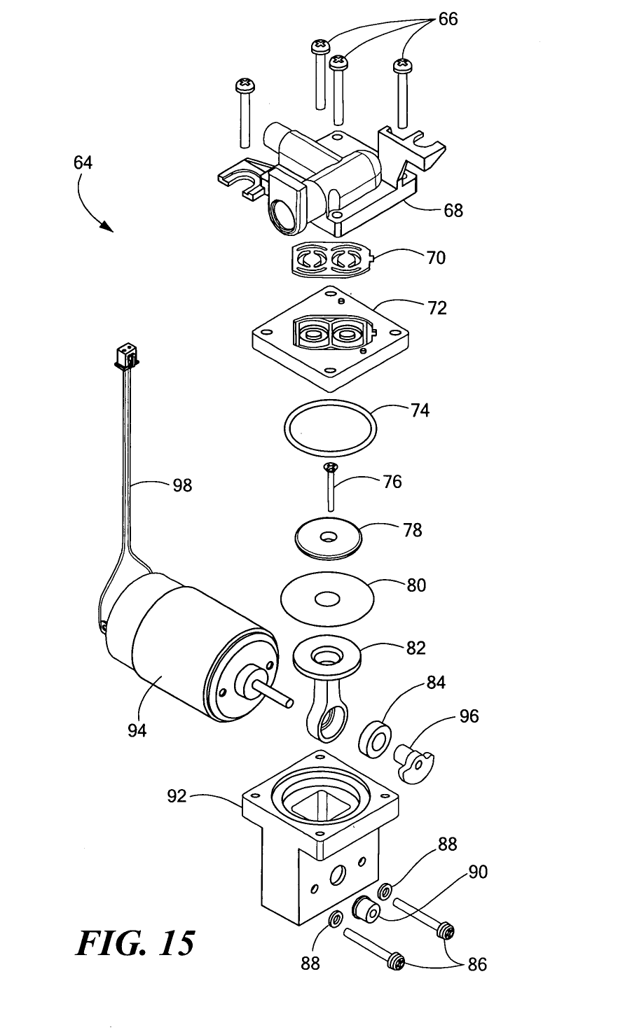

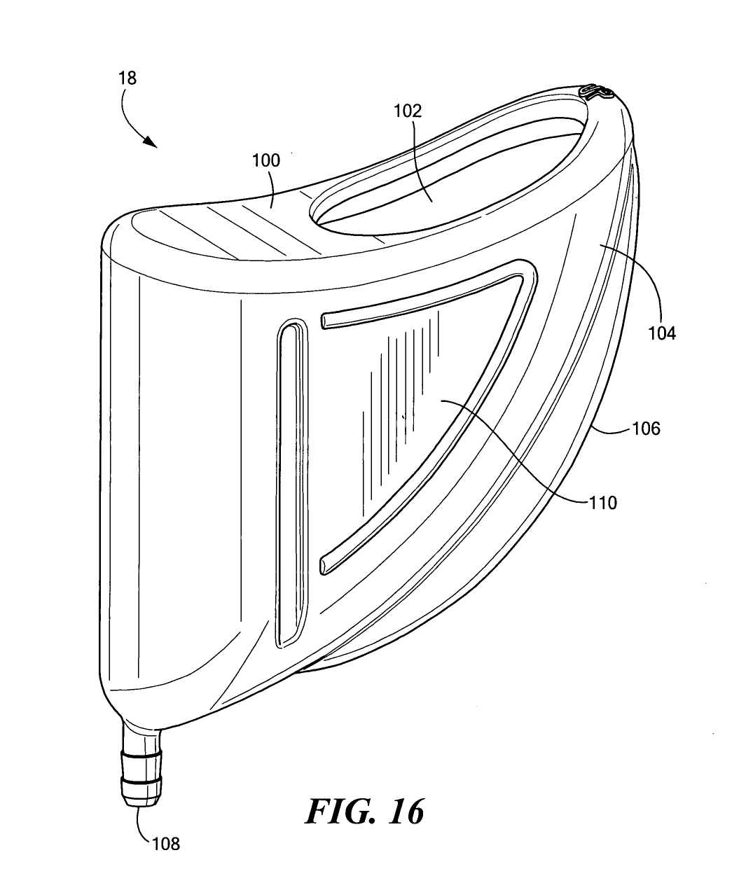

Turning to FIGS. 14 and 15, a line drawing perspective view and an exploded view, respectively, of an example embodiment of a pump assembly 64 suitable for use in the present inventive urine collection system 10 are depicted. The pump assembly 64 includes fasteners 66, pump head 68, pump valve 70, pump chamber 72, o-ring 74, fastener 76, pump piston head 78, pump piston diaphragm 80, pump piston 82, roller bearing 84, fasteners 86, lock washers 88, bushing 90, pump housing 92, motor 94, pump cam offset 96, and pump wiring harness 98. These components are assembled as depicted in FIGS. 14 and 15.

The motor 94 may be implemented using, for example, a commercially available motor model number RS-555PCNVC manufactured by Mabuchi Motor Co., LTD. of Matsuhidai City, Chiba, Japan. This particular motor used in the present application operates at a nominal voltage of 12 volts and at a loaded speed of 1800 to 2000 RPM. Other motors may be used. In some embodiments, the pump may be calibrated to create a flow rate of approximately 1.2 to approximately 1.4 liters/minute. Higher flow rates (e.g., rates greater than 1.4 liters/minute) may be used to remove more urine faster if necessary. Slower flow rates (e.g., rates less than 1.2 liters per minute) may result in the collection receptacle 18 not being drained fast enough to prevent splash-back and eventually overflow. In some embodiments, the flow rate may be adjustable by the user and any rate greater than the rate of urination may be used.

In some embodiments, the maximum exhaust pressure of the pump once it is primed may be approximately 15 PSI to approximately 20 PSI and the maximum vacuum pressure generated by the pump may be approximately 15 to approximately 20 feet of water. Note however that in some embodiments, the pump percussion relief tube 58 may be used to provide a failsafe measure to limit the maximum vacuum pressure, e.g., to approximately 10 feet of water. For example, in some embodiments, approximately 28″ to 30″ of pre-coiled clear plastic tubing with a 0.25″ inner diameter may be used for the conduit tube. Coupled to the end of the clear plastic tubing, inline just before the pump, a percussion relief tube 58 may be inserted as mentioned above. The material used for the percussion relief tube 58 may be selected so as to include a material property such that the tube collapses and closes off the pump inlet when the vacuum pressure exceeds the desired failsafe threshold, e.g., approximately 10 feet of water. Thus, if the one or more vents in the collection receptacle 18 become blocked, the system includes a failsafe measure to prevent any potentially dangerous or harmful buildup of vacuum pressure.

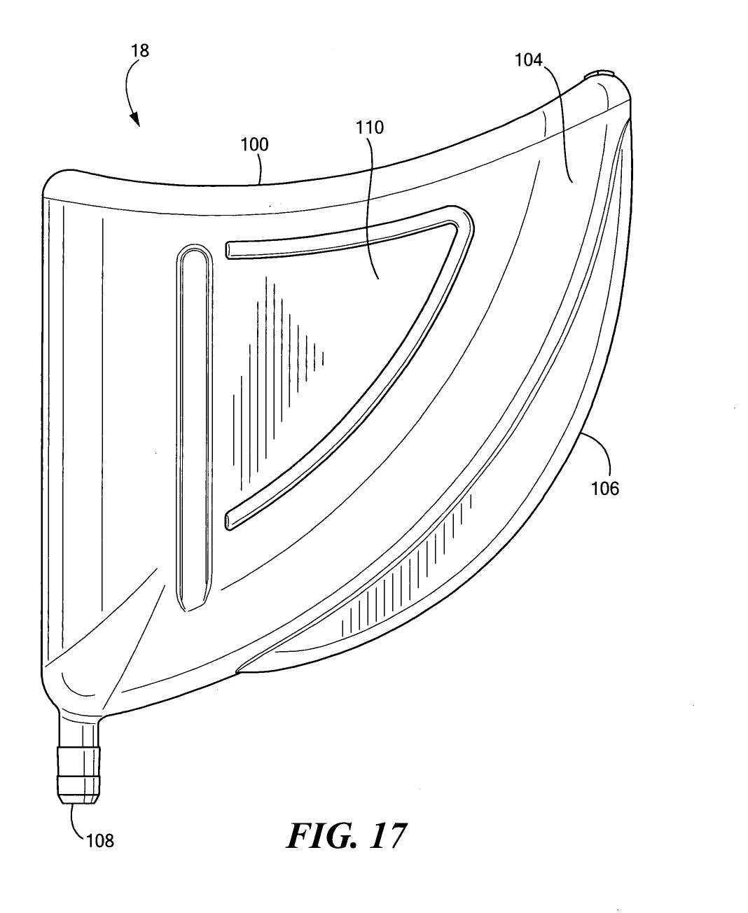

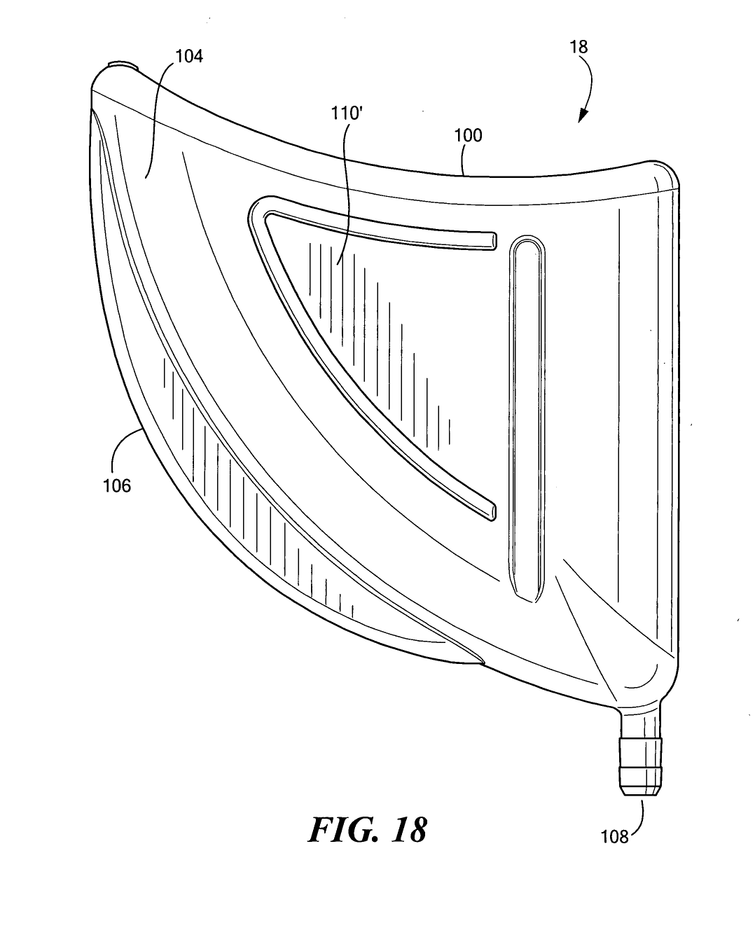

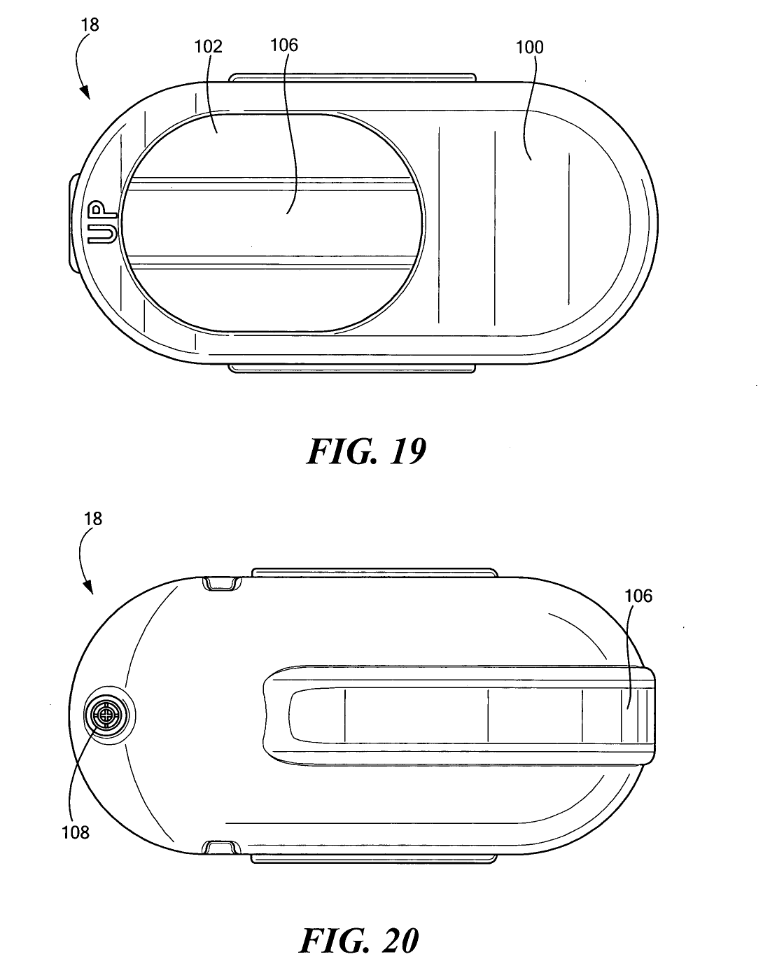

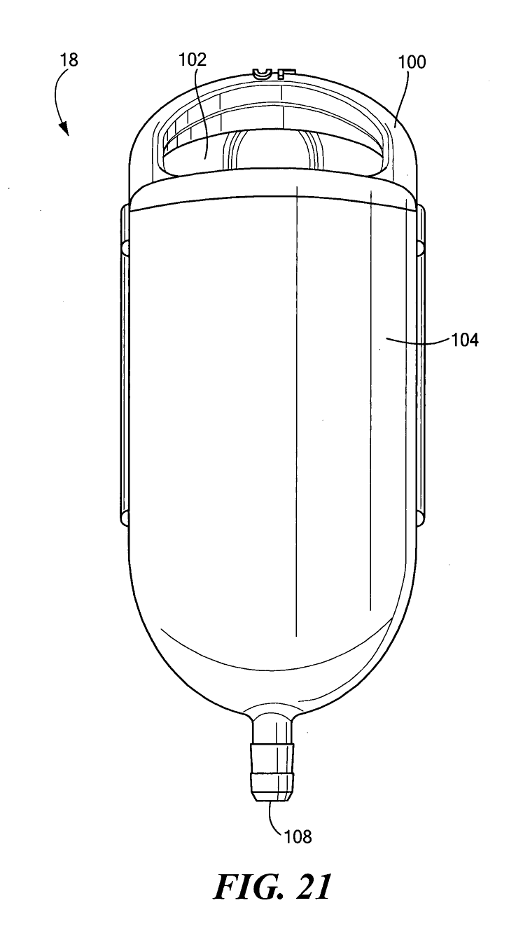

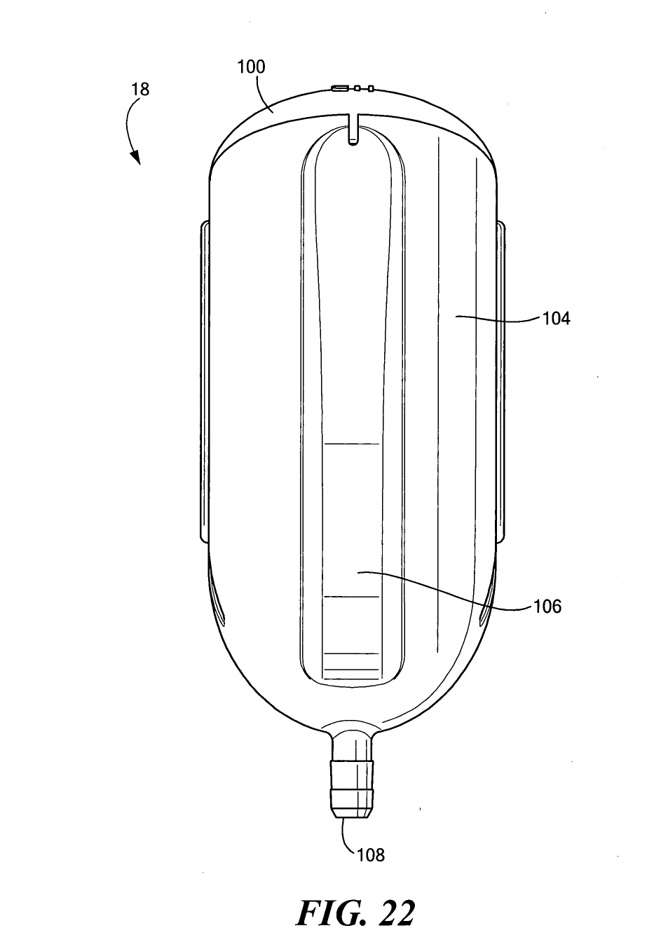

FIGS. 16 through 22 are respective views of an example collection receptacle 18 assembly suitable for use with a urine collection system 10 in accordance with the present invention. The collection receptacle 18 may generally have a receptacle 18 body including a first surface 100 defining a first opening 102 therein for receiving a fluid. The first surface 100 may be contoured and/or concave to ease positioning of the receptacle 18 against an individual for use. In particular, the first surface 100 and the opening are adapted to both be positioned against the labium when used by a female and to receive a penis when used by a male. Although not pictured, in some embodiments, the collection receptacle 18 may include one or more orifices or vents that are positioned to both allow air to freely enter the collection receptacle 18 and to prevent urine from leaking out of the collection receptacle 18. The vents may be of sufficient size to prevent vacuum pressure from building up within the collection receptacle 18. Thus, in such embodiments, the collection receptacle 18 does not develop or require a sealed vacuum coupling with the skin of the user as described in the Kraus reference discussed above.

The collection receptacle 18 900 may further include an interior chamber defined by the first surface 100 and one or more sidewalls 104, where at least one of the sidewalls includes a deep channel 106 extending along a length thereof to receive and redirect a urine stream without creating and/or minimizing splash-back. As used herein, the term “sidewall” is intended to include any surface or wall extending from the first surface 100 to define the interior chamber. The receptacle 18 body may be curved and/or contoured as include a substantially continuous single sidewall. The receptacle 18 body may further define a second opening 108 for fluid to exit the interior chamber. The second opening 108 may be located opposite the first opening 102, and the sidewall 104 may define an arcuate portion extending substantially from the first surface 100 towards the second opening 108. The channel 106 may extend along the arcuate portion of the sidewall 104 to direct fluid towards the second opening 108. As can be seen in FIGS. 16-18, first and second depressed regions 110, 110′ may be included on the at least one sidewall 104, where the depressed regions 110, 110′ are adapted to provide finger holds for securely gripping the collection receptacle 18 900.

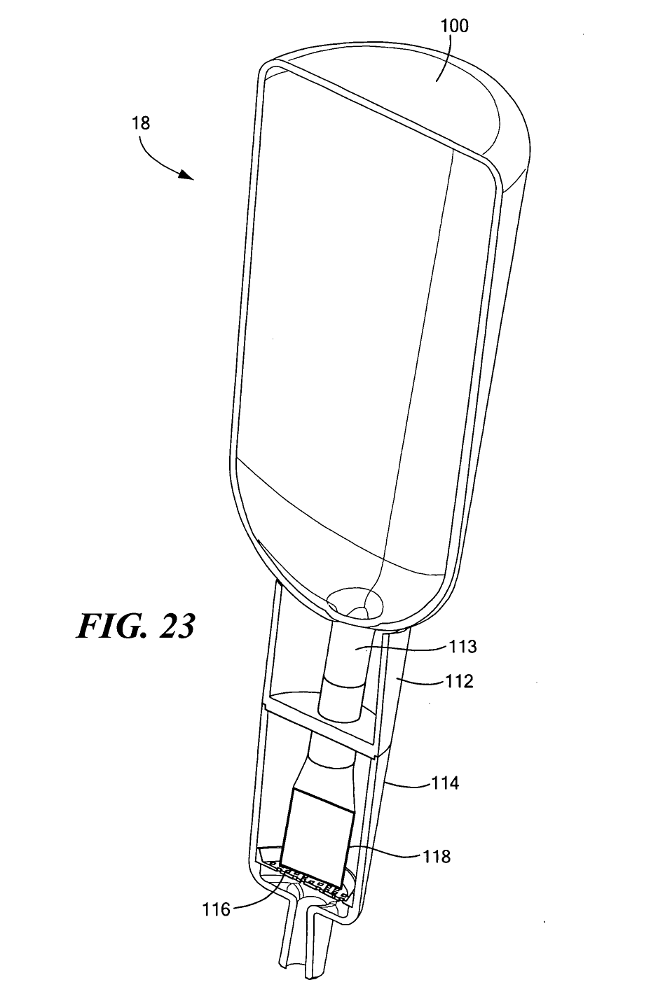

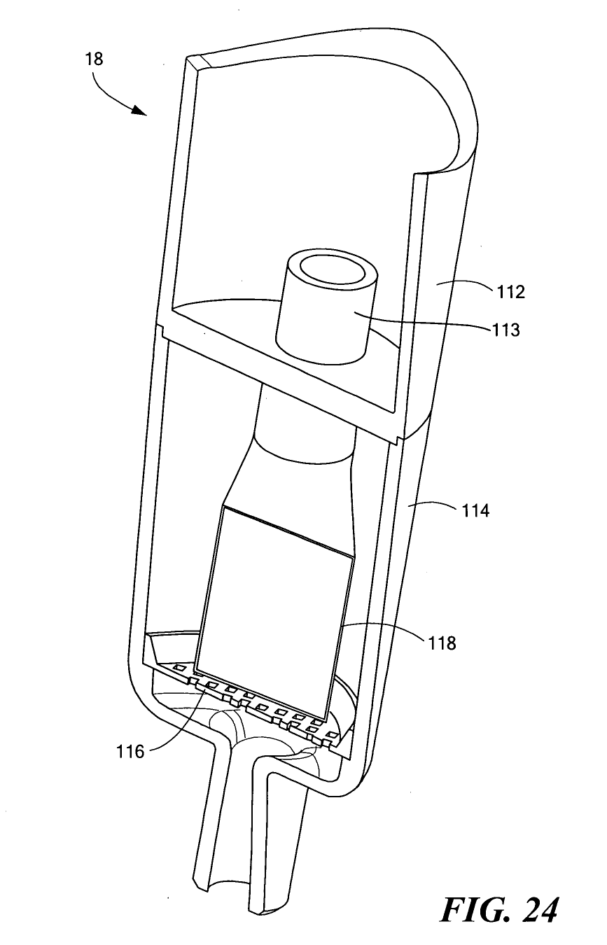

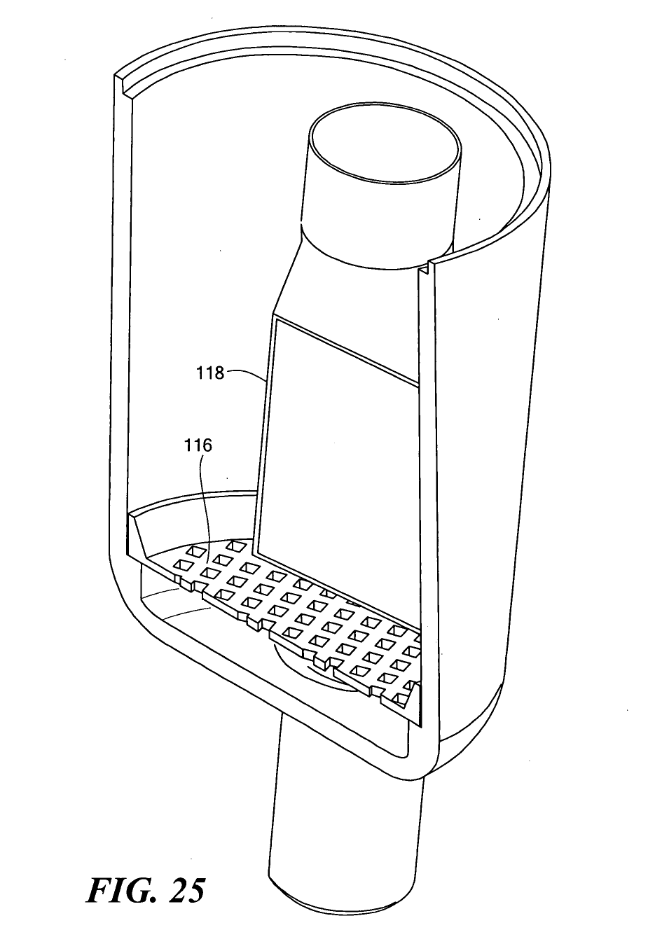

Now referring to FIGS. 23-25, three increasingly magnified cross-sectional perspective views of the component parts of an example collection receptacle assembly are depicted. The collection receptacle assembly includes collection receptacle 18 coupled to, and in fluid communication with, a muffler tube 112 disposed about a portion of a conduit 113, where the conduit 113 is further coupled to and in fluid communication with a valve chamber 114 that is adapted to carry urine and air to the pump assembly 64. The cross-sectional view allows the inside of the muffler tube 112 and the valve chamber 114 to be clearly seen. The muffler tube 112 surrounds the conduit 113, and the valve chamber 114 includes a filter 116 and a reflux valve 118. The filter 116 protects the pump from debris, large particles in the urine, and any other foreign matter that may otherwise enter the pump. The reflux valve 118 prevents undesirable backward flow of urine from the conduit into the collection receptacle 18. The muffler tube 112 in combination with the reflux valve 118 functions to trap noise (e.g., gurgling/slurping sounds that occur as the pump transitions between moving air and moving urine, other pump sounds related to priming and rapidly moving a pump diaphragm, etc.) and helps maintain the quite operation of the urine collection system 10.

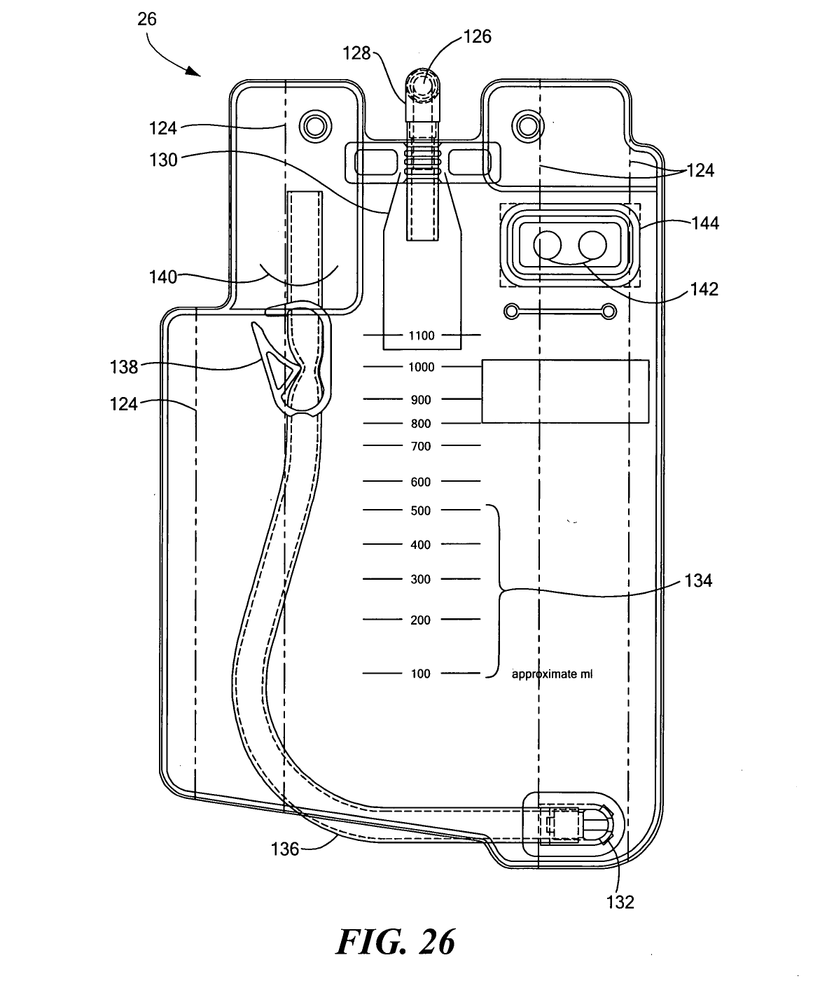

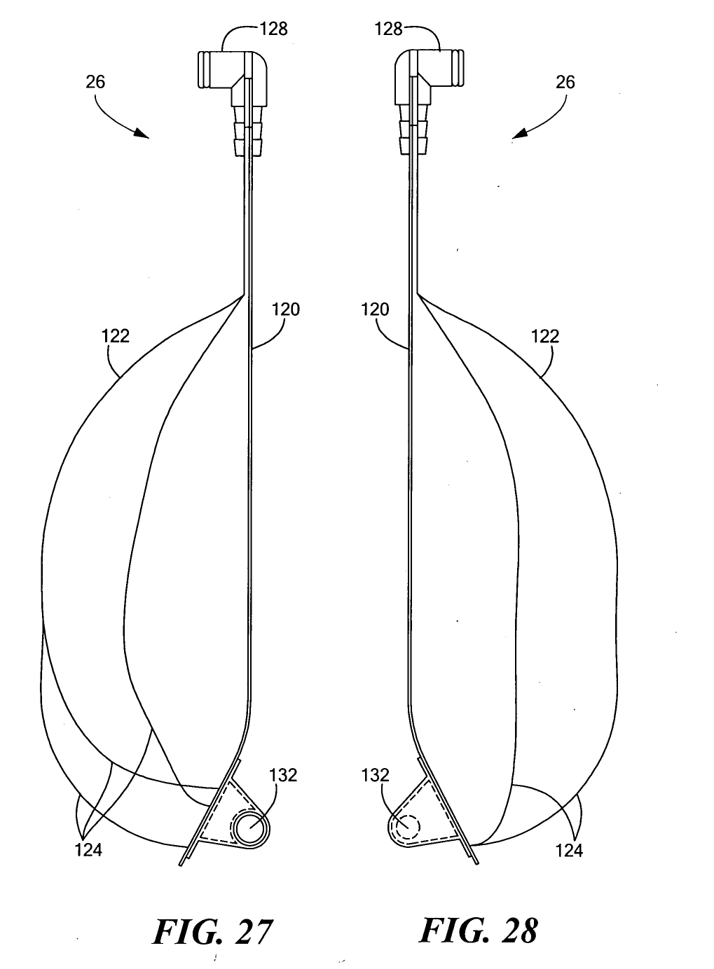

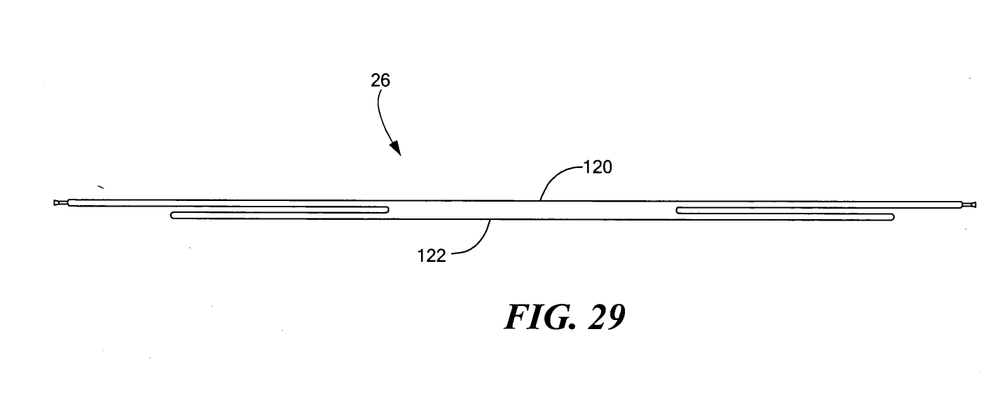

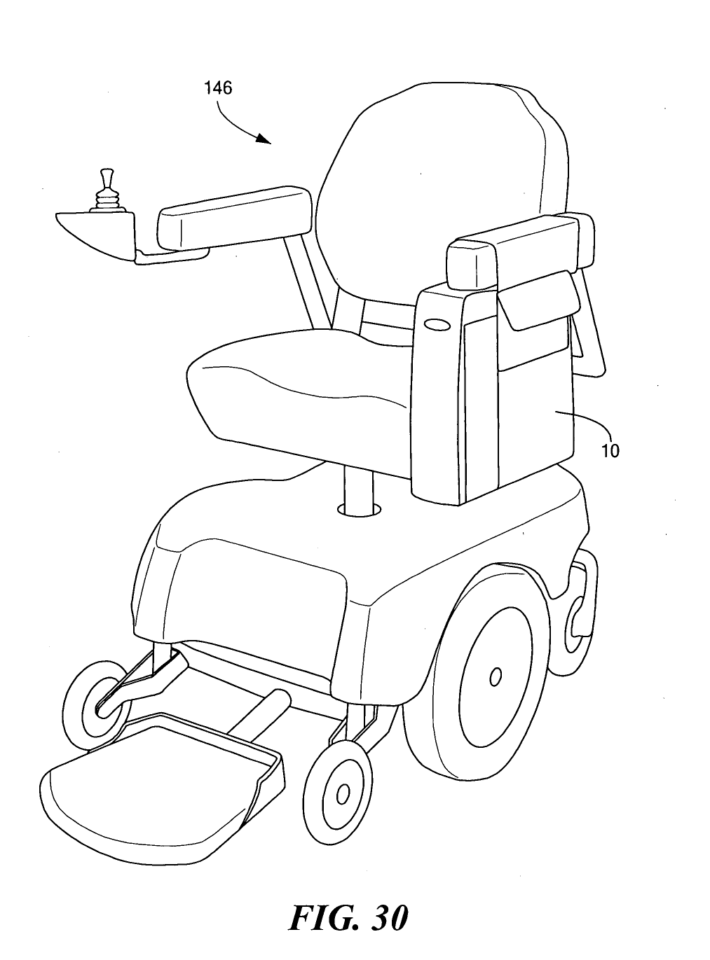

Now referring to FIGS. 26-29, the urine collection reservoir 26 of the present invention is illustrated as being adapted to expand in only one direction as the reservoir 26 is filled. In other words, when empty, the reservoir 26 may hang flat within the housing of the apparatus 10, but as it fills, only one side of the reservoir 26 expands laterally while the other side remains in substantially inexpansible and/or in the same plane as it was when the reservoir 26 was empty. For example, the collection reservoir 26 may generally define or include a first side 120 and a second side 122, where the first and second sides are bonded together to define an interior cavity for receiving and holding a fluid therein. The first side 120 may be constructed from a flat, substantially non-expanding material, while the second side 122 may include one or more expandable pleats or folds 124 in the material, which allow the second side 122 to expand laterally upon filling of the reservoir 26.

Expansion in only one direction may have a number of uses and benefits. For example, in the portable urine collection system 10, the non-expanding side of the reservoir 26 may be mounted flush against an inner wall of the enclosure 12 of the system and the input port, drainage tube, venting/filter port, etc. may be reliably located in a stationary position as opposed to being pushed out as the reservoir 26 fills. In a hospital bed application, the non-expanding side of the reservoir 26 may be disposed so as to face out away from the bed so that as the reservoir 26 fills, the expanding side of the reservoir 26 expands under the bed. This allows the profile of the reservoir 26 to remain flush with the side of the bed even when the reservoir 26 is full. The benefit of the reservoir 26 remaining flush with the bed is that extra clearance is not required for equipment, nurses, doctors, and/or by the patient getting in and out of the bed, even when the reservoir 26 is full. This reduces the chances of the collection reservoir 26 getting bumped and accidentally ruptured or inadvertently punctured or pinched.

The drawings depict embodiments of the reservoir 26 viewed from different sides, which depict the one-way expansion of the collection reservoir 26 as it is filled. In contrast to a reservoir 26 that include expansion pleats on both side panels of the reservoir 26, the present reservoir 26 may include pleats on only one side and further, the bonding of the panels used to form the pleats may be along the edge outside of the pleats as opposed to being pleated on the front and back pleat panel sections themselves and then sealed together along an edge.

The reservoir 26 may further include a first opening 126 in fluid communication with the interior cavity of the reservoir 26, where the first opening 126 may be coupled to the conduit and/or pump described above for receiving urine from the collection receptacle 18. The opening 126 may include an angled connector 128 that may be rotatable and/or able to swivel to ease the coupling of the reservoir 26 to a particular fluid conduit. The collection reservoir 26 may further include a check valve 130 coupled to or otherwise in fluid communication with the first opening 1262 to ensure that fluid only flows into the reservoir 26 while limiting or preventing backflow out of the first opening 126.

In addition to the first opening 126, the collection reservoir 26 may include or define a second opening 132 in fluid communication with the interior cavity of the reservoir 26 to facilitate draining or emptying of the reservoir 26 when a particular fluid level within the reservoir 26 has been reached. A particular fluid level may be assessed by including a fluid level indicator 134 on one and/or both of the first and second sides, where the fluid level indicator 134 may include a graduated scale of fluid measurements corresponding to a particular fluid level in the reservoir 26 (i.e., ml, oz., etc). A length of tubing or a conduit 136 may be coupled to the second opening 132 to aid in directing fluid flow out of the reservoir 26 upon emptying. A clamp or flow limiting element 138 may be affixed to the conduit 136 to ensure that fluid flows through the conduit 136 only when desired or that fluid flow is limited to a desired flow rate. Moreover, at least one of the first or second sides may define a pocket or pouch 140 able to receive at least a portion of the conduit until the reservoir 26 requires emptying.

The fluid collection reservoir 26 may further define a vent 142 or other opening on either and/or both of the first and second sides. The vent 142 may allow excess pressure to released and further reduce the likelihood that the reservoir 26 will rupture or experience a structural failure due to excessive amounts of gas pressure or the like building up in the reservoir 26. The vent may also include a filter 144 that prevents particles of a certain size or the like from escaping the reservoir 26.

FIG. 30 is a perspective view of an embodiment of a urine collection system 10 mounted as an accessory on a powered wheelchair 146. As shown, the material on the top of the enclosure 12 is looped over an arm of the powered wheelchair so that the urine collection system 10 is conveniently and ergonomically mounted adjacent a user seated in the wheelchair. FIG. 30 further illustrates that the mounting position below the arm of the wheelchair and the slim profile of the urine collection system 10 of the present invention allows the system to be mounted without increasing the overall width of the accessorized wheelchair beyond the original width of the powered wheelchair's wheels. Note also that the status/control panel 30 and the swing-out compartment extend just beyond the armrest to facilitate finger tip control of the system and easy access of the collection receptacle 18. In some embodiments, the urine collection system 10 of the present invention may be adapted to be powered by the power supply of the powered wheelchair. In alternative embodiments, the urine collection system 10 of the present invention may be adapted to recharge its batteries using the power supply of the powered wheelchair.

In addition to the coupling of the apparatus to a wheelchair as described above, the material on the top of the enclosure 12 is looped over an arm of a powered scooter or other motorized vehicle so that the urine collection system 10 is conveniently and ergonomically mounted adjacent a user seated in the scooter. In addition, the mounting position below the arm of a scooter and the slim profile of the urine collection system 10 of the present invention allows the system to be mounted without increasing the overall width of a particular accessorized scooter beyond the original width of the powered scooter's wheels. Moreover, the status/control panel and the swing-out compartment may extend just beyond the armrest to facilitate finger tip control of the system and easy access of the collection receptacle 18. In some embodiments, the urine collection system 10 of the present invention may be adapted to be powered by the power supply of the powered scooter. In alternative embodiments, the urine collection system 10 of the present invention may be adapted to recharge its batteries using the power supply of the powered scooter.

Although not illustrated, it is understood that the compact and convenient nature of the urine collection system 10 may allow it to be coupled to a myriad of personal vehicles, including wheelchairs and the like. For example, the apparatus may be suspended from a swing-arm mounted on a mechanical wheelchair. The swing arm may be adapted to store the urine collection system 10 of the present invention out of the way, behind the back of the wheelchair. When the user needs the system, a lever may be pulled that swings the system out from behind the wheelchair into a position adjacent the user. When the user has completed his or her use of the system, the system may be swung back behind the wheelchair with a push of the lever. Such a lever driven swing-arm may be adapted to be mounted on any type of chair or scooter. The invention allows a user immediate and convenient access to the system without having to get up from the chair, and the system can be moved away from the wheels when not in use.

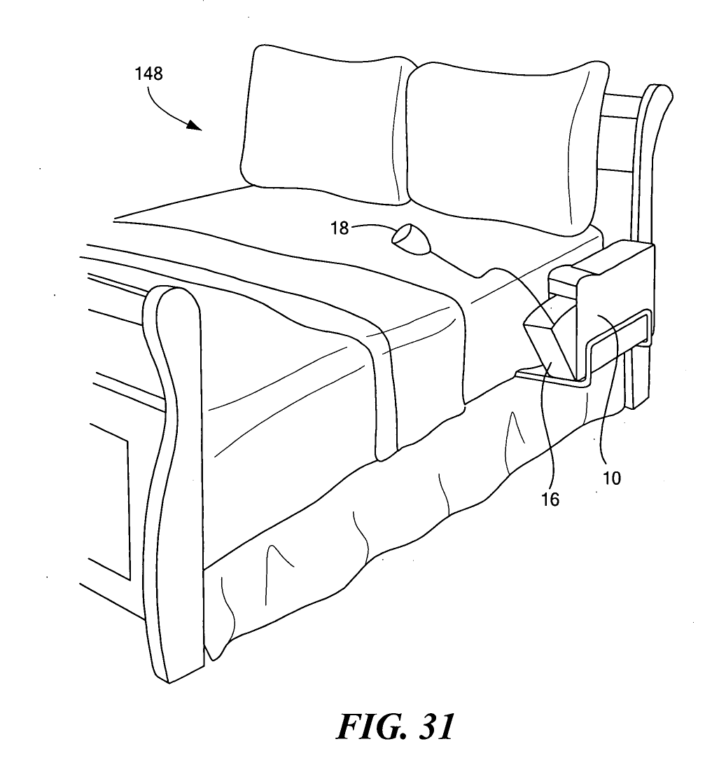

FIG. 31 depicts a perspective view of an embodiment of a urine collection system 10 supported by a frame mounted on a bed 148. The frame may include a bracket adapted to securely hold or clamp the system in a convenient location and height. The frame may also include extended members that fit under the mattress and/or box spring such that the frame is securely held in place by the weight of the mattress and/or box spring.

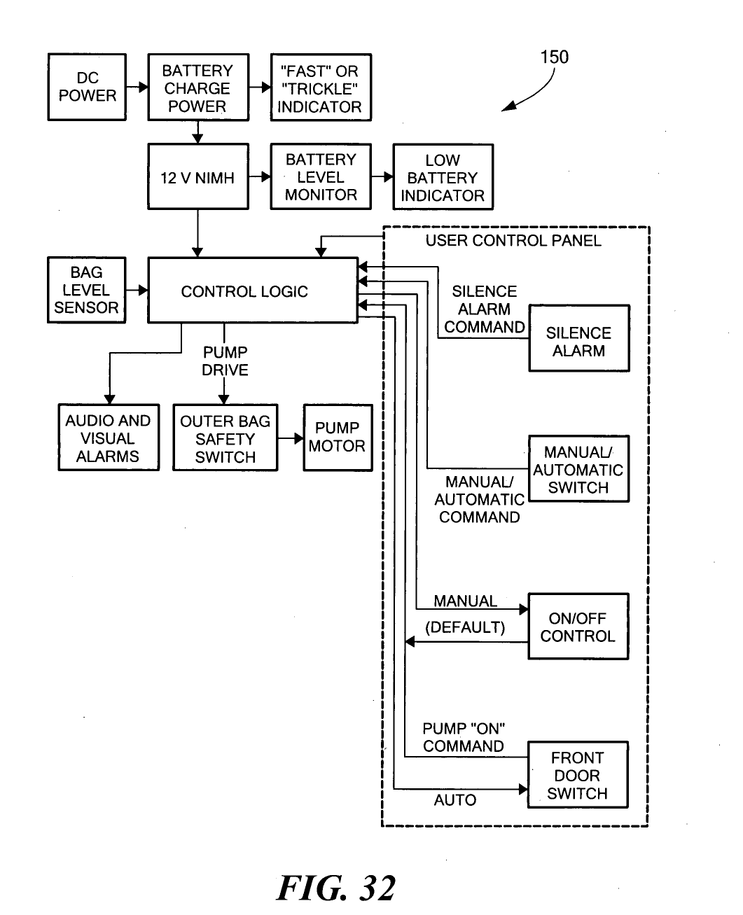

FIG. 32 depicts a block diagram of an embodiment of a urine collection system 10 control circuit 150. A pre-programmed processor and/or a programmable gate array may be used to implement a central control logic. In alternative embodiments, discrete logic may be used. The control logic may be adapted to receive control signals from the user control panel which includes three switches: the silence alarm switch; the manual/auto select switch; and the pump on/off control switch. The pump on/off switch is enabled by the control logic based on the state of the manual/auto select switch. In addition to the control signals from the user control panel, the control logic is coupled to and adapted to receive pump control signals from a front door 16 (or swing-out compartment) switch (which is enabled by the control logic based on the state of the manual/auto select switch) and a reservoir 26 fullness level sensor. The control logic can generate a pump drive signal in response to the pump control signals but the pump drive signal is gated by the state of the enclosure 12 safety (access door 16) switch. The control logic is also able to generate various control signals for activating audio visual alarms and status LEDs. The control logic receives a power signal from the batteries which are monitored by a battery level meter which outputs a low battery signal to a low battery indicator A/V alarm (e.g., LED or beep tone). The batteries receive DC power for charging via a battery charge power circuit based on whether the DC power is connected to a power source. When connected, the battery charge power circuit determines whether the batteries receive a “FAST” charge or a “TRICKLE” charge and the FAST or TRICKLE indicator reflects the determination based on a signal from the battery charge power circuit.

The system of the present invention advantageously provides a frame adapted to house a compact rechargeable battery-powered pump and circuitry, concealed within a slim-profile, washable, weather-proof, and durable enclosure 12 (e.g., a soft cover). The system further includes a collection cup adapted for both males and females, drainage tubing, a high-volume reservoir 26, and the vacuum pump which is adapted to operate quietly, safely, and at a vacuum pressure optimally calibrated for removing urine from the user. The enclosure 12 conceals the light weight system (e.g., less than approximately 3 lbs. when empty) and helps muffle the pump while it operates. The system is further adapted to be held, attached to a manual wheelchair or powered scooter, attached to a bed, and/or placed next to an easy chair. In operation, a patient simply voids into a hand-held collection cup and a pump creates a gentle vacuum which draws the urine through a tube into a plastic drainage reservoir 26 where it is stored until emptied. Compared to prior art devices that require immediate disposal, the system of the present invention provides sufficient volume storage for a day's worth of urine before emptying, thereby eliminating stress and urgency for the user to find a toilet.

The present invention offers several additional advantages over prior art devices, including (i) diminished risk of skin irritation, rashes and sores because moisture content near the user is reduced; (ii) reduction in the high incidence of infection and resulting costs of treatment because the device is external and self-applied; (iii) sufficient volume storage for a day's worth of urine before discarding (as opposed to other alternative that require immediate disposal); (iv) reduction in the instances of “slip and fall” accidents, as the device will eliminate the necessity of the users to transfer locations or rush to the bathroom; and (v) ease of use and maintenance compared to other prior art devices.

Regarding maintenance, the system of the present invention may be easily sanitized by periodically allowing a cleaning solution (e.g., vinegar and water) to be pumped though the system. In addition, sprayable odor eliminating solutions may be used as needed. The outer enclosure of the system may be easily washed with a basic cleaning solutions (e.g., soap and water). The system may also include an extended life, rechargeable battery (e.g., the battery may operate for two or more weeks per charge). In some embodiments, an integrated “smart” recharging circuit may be included that both protects ihe battery from overcharging and conditions the battery as needed during recharge.

The present invention provides a simple and cost-effective solution for UI suffers who do not require internal catheterization. The system of the present invention was designed as a simple alternative to the in-dwelling catheter, condom catheter, diaper and bed pan. The methods and systems of the present invention are particularly well suited for use by patients with limited mobility, post-operative surgery patients, the elderly (e.g., people ages 65 and over, the Medicare population with “urge incontinence” needs), spinal cord injury (“SCI”) patients; and disabled/chronic condition patients who are non- or partially ambulatory.

High-level clinical benefits of the present invention include a lower risk of bladder cancer and infections in SCI patients due to the system being an external, self-applied apparatus rather than an inserted catheter. Further, the present invention provides a decreased risk of accidental slips and falls by patients who may be physically unstable or medicated but will not wait for a caretaker to assist with going to the restroom. In addition, many incontinent patients experience continuing exposure to spilled urine increased risk of skin irritation, rashes, and sores. The system of the present invention is operative to draw urine from the patient, thereby decreasing these conditions. From an economic perspective, the present invention provides a cost-effective solution (i.e., simple, relatively inexpensive, discreet, urine collection and stored drainage) that by design is affordable to average consumers and healthcare organizations. Further, some embodiments of the present invention are adapted to utilize consumable components that are currently commercially available and/or covered as “reimbursable items” (e.g., urine collection receptacle 18s, conduit tubing, and reservoir 26s) under Federal healthcare programs such as, e.g., Medicare and Medicaid. Further, prior art urine collection devices overwhelmingly require techniques or assistance typically by trained healthcare professionals. The urine collection system 10 of the present invention empowers non-ambulatory and disabled patients (e.g., both male and female) to be self-sufficient due to the system's simplicity, portability, non-invasiveness, and discreet design.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A urine collection receptacle, comprising:
a receptacle body defining a first surface and at least one sidewall extending from the first surface;
wherein the at least one sidewall and the first surface define an interior chamber;
wherein the first surface defines a first opening in fluid communication with the interior chamber;
wherein the receptacle body further defines a second opening opposite the first opening;
wherein the second opening is in fluid communication with the interior chamber; and
wherein the at least one sidewall defines a channel extending along a length thereof.

2. The urine collection receptacle according to claim 1, wherein the first surface is contoured.

3. The urine collection receptacle according to claim 2, wherein the first surface defines a concave portion.

4. The urine collection receptacle according to claim 1, wherein the at least one sidewall defines an arcuate portion extending from the first surface to the second opening.

5. The urine collection receptacle according to claim 4, wherein the channel extends along a substantial length of the arcuate portion of the sidewall.

6. The urine collection receptacle according to claim 1, wherein the at least one sidewall defines a first depressed region to aid in securely gripping the receptacle.

7. The urine collection receptacle according to claim 6, wherein the at least one sidewall defines a second depressed region to aid in securely gripping the receptacle.

8. The urine collection receptacle according to claim 1, further comprising a conduit in fluid communication with the interior chamber.

9. The urine collection receptacle according to claim 8, further comprising a muffler tube disposed about at least a portion of the conduit.

10. The urine collection receptacle according to claim 1, further comprising a check valve in fluid communication with the interior chamber.

11. The urine collection receptacle according to claim 1, further comprising a filter in fluid communication with the interior chamber.

12. The urine collection receptacle according to claim 1, further comprising a pump coupled to the receptacle body, and a reservoir coupled to the pump, wherein the reservoir is in fluid communication with the interior chamber.

13. The urine collection receptacle according to claim 12, wherein the reservoir includes a first side and a second side, wherein the first side is bonded to the second side to define an interior cavity, and wherein the second side is laterally expandable and the first side is substantially inexpansible when the collection reservoir is filled.

14. A urine collection receptacle, comprising:
a receptacle body defining a contoured first surface and at least one sidewall extending from the first surface, wherein the at least one sidewall and the first surface define an interior chamber;
wherein the first surface defines a first opening in fluid communication with the interior chamber;
wherein the receptacle body further defines a second opening opposite the first opening, the second opening being in fluid communication with the interior chamber;
wherein the at least one sidewall defines an arcuate portion extending from the first surface to the second opening; and
wherein the arcuate portion defines a channel extending along a substantial length thereof.

15. The urine collection receptacle according to claim 14, wherein the at least one sidewall defines a first depressed region to aid in securely gripping the receptacle.

16. The urine collection receptacle according to claim 15, wherein the at least one sidewall defines a second depressed region to aid in securely gripping the receptacle.

17. The urine collection receptacle according to claim 14, further comprising a conduit in fluid communication with the interior chamber.

18. The urine collection receptacle according to claim 17, further comprising a muffler tube disposed about at least a portion of the conduit.

19. The urine collection receptacle according to claim 14, further comprising a check valve in fluid communication with the interior chamber.

20. The urine collection receptacle according to claim 14, further comprising a filter in fluid communication with the interior chamber.

21. A urine collection receptacle, comprising:
a receptacle body defining a contoured first surface and at least one sidewall extending from the first surface, wherein the at least one sidewall and the first surface define an interior chamber;
wherein the first surface defines a first opening in fluid communication with the interior chamber;
wherein the receptacle body further defines a second opening opposite the first opening, the second opening being in fluid communication with the interior chamber;
wherein the at least one sidewall defines an arcuate portion extending from the first surface to the second opening;
wherein the arcuate portion defines a channel extending along a substantial length thereof;
a conduit in fluid communication with the interior chamber;
a muffler tube disposed about at least a portion of the conduit;
a check valve in fluid communication with the interior chamber; and
a filter in fluid communication with the interior chamber.
